(19) United States
(12) Patent Application Publication
(10) Pub. No.: US 2002/0086007 A1
(43) Pub. Date: Jul. 4, 2002

(54) Angiogenesis-inhibiting peptides and proteins and methods of use

(76) Inventors: Kim Lee Sim, Gaithersburg, MD (US); Hong Liang, Gaithersburg, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/978,531

(22) Filed: Oct. 15, 2001

Related U.S. Application Data
(60) Provisional application No. 60/240,127, filed on Oct. 13, 2000.

(51) Int. Cl. A61K 38/52

(52) U.S. Cl. 424/94.5

(57) ABSTRACT

Methods and compositions comprising proteins and mimotopes that are related to or derived from endostatin and PITSLRE protein kinases are provided. The compositions of the present invention comprise proteins that are involved in cell cycle regulation and angiogenesis.

### CROSS REFERENCE TO RELATED APPLICATIONS

This application claims priority to U.S. Provisional Application Serial No. 60/240,127 filed Oct. 13, 2000.

### FIELD OF THE INVENTION

The present invention is related to compositions and methods for the modulation of angiogenesis. In particular, the present invention comprises peptides, proteins and mimotopes (conformational structures), that are related to endostatin, PITSLRE protein kinases and combinations thereof. The proteins, peptides and mimotopes of the present invention are capable of modulating angiogenesis through various mechanisms such as the regulation of transcription during the cell cycle or the triggering of the apoptotic signaling pathway.

### BACKGROUND OF THE INVENTION

As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonal development and formation of the corpus luteum, endometrium and placenta. The term “endothelium” means a thin layer of flat epithelial cells that lines serous cavities, lymph vessels, and blood vessels.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops thereby creating the new blood vessel.

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together as angiogenic-dependent or angiogenic-associated diseases.

The hypothesis that tumor growth is angiogenesisdependent was first proposed in 1971 (Folkman J., Tumor angiogenesis: Therapeutic implications., N. Engl. Jour. Med. 285:1182 1186, 1971). In its simplest terms it states: “Once tumor ‘take’ has occurred, every increase in tumor cell population must be preceded by an increase in new capillaries converging on the tumor.” Tumor “take” is currently understood to indicate a prevascular phase of tumor growth in which a population of tumor cells occupying a few cubic millimeters volume and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

It is clear that angiogenesis plays a major role in the metastasis of a cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

One example of a disease mediated by angiogenesis is ocular neovascular disease. This disease is characterized by invasion of new blood vessels into the structures of the eye such as the retina or cornea. It is the most common cause of blindness and is involved in approximately twenty eye diseases. In age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegener's sarcoidosis, Scleritis, Steven's Johnson disease, periphigoid radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, trauma and post-laser complications. Other diseases include, but are not limited to, diseases associated with rubeosis (neovascularization of the eye) and diseases caused by the abnormal proliferation of fibrovascular or fibrous tissue including all forms of proliferative vitreoretinopathy.

Another disease in which angiogenesis is believed to be involved is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Factors associated with angiogenesis may also have a role in osteoarthritis. The activation of the chondrocytes by angiogenic-related factors contributes to the destruction of the joint. At a later stage, the angiogenic factors would promote new bone formation. Therapeutic intervention that prevents the bone destruction could halt the progress of the disease and provide relief for persons suffering with arthritis.

Chronic inflammation may also involve pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy resistant hemangiomas exist that cannot be treated with therapeutics currently in use.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula.

Numerous efforts have been made by researchers in the pharmaceutical industry to improve the target specificity of drugs. As is familiar to those skilled in the art, the manifestation of a disease many times involves the display of a particular cell type or protein as an antigenic, epitopic, or surface marker. In such instances, an antibody can be raised against the unique cell surface marker and a drug can be linked to the antibody. Upon administration of the drug/antibody complex to a patient, the binding of the antibody to the cell surface marker results in the delivery of a relatively high concentration of the drug to the diseased tissue or organ. Similar methods can be used where a particular cell type in the diseased organ expresses a unique cell surface receptor or a ligand for a particular receptor. In these cases, the drug can be linked to the specific ligand or to the receptor, respectively, thus providing a means to deliver a relatively high concentration of the drug to the diseased organ.

Endostatin

An important angiogenesis-related protein is endostatin protein (see U.S. Pat. No. 5,854,205 and WO 97/15666 O'Reilly et al., both of which are incorporated herein by reference in their entirety). Endostatin protein is a potent and specific inhibitor of endothelial proliferation and angiogenesis. Systemic therapy with endostatin protein causes a nearly complete suppression of tumor induced angiogenesis.

One form of the endostatin protein has a molecular weight of approximately 18,000 to approximately 20,000 Daltons as determined by non-reduced and reduced gel electrophoresis, respectively, and is capable of inhibiting endothelial cell proliferation in cultured endothelial cells. Endostatin protein has an amino acid sequence substantially similar to a fragment of a collagen molecule and whereas it binds to a heparin affinity column, it does not bind to a lysine affinity column.

Endostatin protein can be isolated from murine hemangioendothelioma EOMA. Endostatin protein may also be produced from recombinant sources, from genetically altered cells implanted into animals, from tumors, and from cell cultures as well as other sources. Endostatin protein can be isolated from body fluids including, but not limited to, serum and urine. Recombinant techniques including, but not limited to, gene amplification from DNA sources using the polymerase chain reaction (PCR), and gene amplification from RNA sources using reverse transcriptase/PCR can be used to clone the endostatin gene into an expression vector to express recombinant endostatin.

Alternatively, endothelial proliferation inhibiting proteins, or endostatin proteins may be isolated from larger known proteins, such as human alpha 1 type XVIII collagen and mouse alpha 1 type XVIII collagen, proteins that share a common or similar N-terminal amino acid sequence. Examples of other potential endostatin protein source materials having similar N-terminal amino acid sequences include Bos taurus pregastric esterase, human alpha 1 type XV collagen, AND-dependent formate dehydrogenase (EC 1.2.1.2) derived from Pseudomonas sp., s11459 hexon protein of bovine adenovirus type 3, CELF21D12 2 F21d12.3 Caenorhabditis elegans gene product, VAL1 TGMV AL1 protein derived from tomato golden mosaic virus, s01730 hexon protein derived from human adenovirus 12, and Saccharomyces cerevisiae.

Human endostatin can be further characterized by its preferred amino acid sequence as set forth in FIG. 2 and in SEQ ID NO: 1. The preferred sequence of the first 20 amino-terminal amino acids corresponds to a C-terminal fragment of collagen type XVIII or collagen type XV. Specifically, in one embodiment the amino terminal amino acid sequence of endostatin protein corresponds to an internal 20 amino acid peptide region found in mouse collagen alpha 1 type XVIII starting at amino acid 1105 and ending at amino acid 1124. The amino terminal amino acid sequence of the inhibitor also corresponds to an internal 20 amino acid peptide region found in human collagen alpha 1 type XVIII starting at amino acid 1132 and ending at amino acid 1151. The cDNA sequence for endostatin protein is provided as SEQ ID NO: 2.

Endostatin protein specifically and reversibly inhibits endothelial cell proliferation and may be used, for example, as a birth control drug, for treating angiogenesis-related diseases, particularly angiogenesis-dependent cancers and tumors, and for curing angiogenesis-dependent cancers and tumors. Therapies directed at control of the angiogenic processes could lead to the abrogation or mitigation of such diseases mediated by angiogenesis. Potential therapies useful for controlling angiogenic processes may involve recognition of antigenic markers and receptors associated with angiogenesis and subsequent modification of such markers and receptors. For example, once a receptor for an angiogenesis-related protein is identified, it can be blocked, thereby inhibiting the effect of the angiogenesis-related protein and ultimately reducing angiogenesis.

Although it has been shown that endostatin is a potent inhibitor of angiogenesis, and can therefore be used for the treatment of angiogenesis-related diseases such as cancer, what is needed in the art is the identification of endostatin binding proteins and peptides. Identification of endostatin binding proteins would allow for the further elucidation of the mechanism of action of endostatin. Further elucidation of the mechanism of action of endostatin would allow for the creation of endostatin mimetics and for the creation of compositions and methods for the inhibition of angiogenesis that in essence “by-pass” the point of action of endostatin.

PITSLRE Protein Kinases

The PITSLRE proteins are part of the large family of p34^{cdc2} related kinases whose functions appear to be linked to control of cell division and possibly programmed cell death. (See, for example, Proteolytic Activities that Mediate Apoptosis, Annu. Rev. Physiol. 1998, 60:533-73) PITSLRE is a large subfamily of protein kinases, with at least 20 isoforms generated by alternative slicing from 3 duplicated and tandemly arrayed human genes (PITSLRE A, B, C). Structurally, PITSLRE protein isoforms contain an N-terminal amino acid domain, catalytic domain and C-terminal region. The catalytic domain and the carboxyterminal of these kinases are essentially conserved. Most of the differences between these various PITSLRE isoforms are found at the N-terminal end of the molecule. PITSLRE homologues have been identified in mouse, chicken, Drosophila, Xenopus, and possibly Plasmodium falciparum, suggesting that their function may be well conserved.

PITSLRE genes are located at chromosome I p36. There are some other apoptosis related genes are located at this region, such as DR3, DR3L, TNFR2, CD3O, 0X40, and the 4-1BB ligand which all encode proteins related to the TNF/NGF death-receptor family and their pathways; a novel p53 related gene, p73; and MMP21-22 (Metalloprotease) gene. The chromosome 1 p36.3 has frequently been found to be deleted or frequently in ductal carcinoma of the breast, neuroplastoma (a subset of malignant melanoma), Merkel cell carcinoma, colon carcinoma.

The functions of PITSLRE protein kinases have been postulated to be involvement in the regulation of RNA splicing/transcription during the cell cycle; and/or involvement in apoptotic signal pathways. It is evident that the PITSLRE protein kinase family plays an important role in regulation of cell growth. What is not known however, is the specific mechanism of action of PITSLRE protein kinases, whether they are involved in angiogenesis and whether they have any interaction with angiogenic factors (inhibitors or stimulators).

Given the important role of angiogenic factors such as endostatin in treating angiogenic-related disease such as cancer, what is needed is the identification and characterization of possible interations between such factors and factors involved in cell cycle regulation such as PITSLRE protein kinases. Furthermore, what is needed, is the development of methods and compositions for the identification of receptors and molecules that bind such proteins. The identification of such receptors and molecules would facilitate the understanding of angiogenesis-related protein influence and interaction, and consequently enable the development of drugs to modify the activity of these proteins as necessary. In addition, the identification of specific fragments of PITSLRE protein kinases that are involved in the regulation of angiogenesis would provide important therapeutic and diagnostic agents.

### SUMMARY OF THE INVENTION

The present invention comprises methods and compositions comprising peptides, proteins and mimotopes that are related to endostatin, PITSLRE protein kinases and combinations thereof. The proteins, peptides and mimotopes of the present invention are capable of modulating angiogenesis through various mechanisms such as, for example, the regulation of transcription during the cell cycle or the triggering of the apoptotic signaling pathway.

The methods of the present invention include methods of increasing angiogenesis in an individual comprising administering an angiogenesis increasing amount of an endostatin binding protein wherein the binding protein comprises proteins related to PITSLRE protein kinases and/or active fragments thereof. The present invention still further includes methods of inhibiting angiogenesis in an individual comprising administering to the individual an angiogenesis inhibiting amount of an endostatin binding protein wherein the binding protein comprises proteins related to PITSLRE protein kinases and/or active fragments thereof. Also included are mimetics and isoforms of the binding proteins.

The present invention further includes the nucleotide sequences encoding the peptides and proteins of the invention related to endostatin and PITSLRE protein kinases, as well as expression vectors containing nucleotide sequences encoding such binding peptides and proteins, and cells containing one or more expression vectors containing nucleotide sequences encoding such peptides and proteins. The invention further encompasses gene therapy methods whereby nucleotide sequences encoding angiogenesis-related protein binding peptides and proteins are introduced into a patient to modify in vivo endostatinprotein levels.

The present invention also includes diagnostic methods and kits for detection and measurement of peptides and proteins that bind angiogenesis-related proteins in biological fluids and tissues, and for localization of such peptides and proteins in tissues and cells. The diagnostic method and kit can be in any configuration well known to those of ordinary skill in the art.

The present invention includes peptides and proteins that bind angiogenic factors such as endostatin protein and cause the transmission of an appropriate signal to a cell and act as agonists or antagonists of angiogenesis.

In addition, the present invention includes fragments of proteins that bind angiogenesis-related proteins, and analogs thereof, that can be labeled isotopically, or with other molecules or proteins, for use in the detection and visualization of angiogenesis-related protein binding sites with techniques, including, but not limited to, positron emission tomography, autoradiography, flow cytometry, radioreceptor binding assays, and immunohistochemistry.

The peptides and analogs of the present invention also act as agonists and antagonists for endostatin protein receptors, thereby enhancing or blocking the biological activity of endostatin protein. Such peptides and proteins are used in the isolation of endostatin protein receptors.

The present invention includes molecular probes for the ribonucleic acid and deoxyribonucleic acid involved in transcription and translation of angiogenesis-related protein binding peptides and proteins. These molecular probes provide means to detect and measure angiogenesis-related protein biosynthesis in tissues and cells.

Accordingly, it is an object of the present invention to provide compositions and methods comprising peptides and proteins that regulate angiogenesis.

It is another object of the present invention to provide compositions and methods for treating diseases and processes that are mediated by angiogenesis.

It is yet another object of the present invention to provide diagnostic or prognostic methods and kits for detecting the presence and amount of angiogenesis-related protein binding peptides in a body fluid or tissue.

Another object of the present invention to provide compositions and methods comprising proteins that are related to PITSLRE protein kinases and are involved in cell cycle regulation.

Yet another object of the present invention to provide compositions and methods comprising proteins that are related to endostatin and PITSLRE protein kinases that are involved in cell cycle regulation and angiogenesis.

A further object of the present invention to provide compositions and methods comprising proteins that are related to endostatin and PITSLRE protein kinases that are involved in cell cycle regulation wherein such proteins comprise structural mimotopes.

It is yet another object of the present invention to provide compositions and methods comprising proteins that are related to endostatin and PITSLRE protein kinases that are involved in angiogenesis.

Another object of the present invention is to provide compositions and methods comprising proteins that are related to endostatin and PITSLRE protein kinases for treating diseases and processes that are mediated by angiogenesis.

It is yet another object of the present invention to provide diagnostic or prognostic methods and kits for detecting the presence and amount of angiogenesis-related protein binding peptides in a body fluid or tissue.

It is yet another object of the present invention to provide compositions and methods for treating diseases and processes that are mediated by angiogenesis including, but not limited to, hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, and cat scratch fever.

It is another object of the present invention to provide compositions and methods for treating or repressing the growth of a cancer.

It is yet another object of the present invention to provide a therapy for cancer that has minimal side effects.

Another object of the present invention is to provide proteins, and fragments thereof, that function as substrates through which angiogenesis-related proteins exert their activities.

Another object of the present invention is to provide methods and compositions for targeted delivery of angiogenesis-related protein compositions to specific locations.

Yet another object of the invention is to provide compositions and methods useful for gene therapy for the modulation of angiogenic processes.

These and other objects, features and advantages of the present invention will become apparent after a review of the following detailed description of the disclosed embodiments.

### BRIEF DESCRIPTION OF THE FIGURES

FIGS. 1a and 1b show sequence alignment of endostatin protein (SEQ ID NO: 3) a representative PITSLRE protein kinase (SEQ ID NO: 4).

FIG. 2a provides the amino acid sequence of one form of endostatin (SEQ ID NO: 1).

FIG. 2b provides the cDNA sequence of one form of endostatin (SEQ ID NO: 2).

FIGS. 3a and 3b show western blots analyzing E. coli lysate of the two PITSLRE protein kinase cDNA clones 2111 and clone 3111.

FIG. 4 shows western blots analyzing the immunoprecipitate endostatin protein from extracted nuclei.

FIG. 5 shows a PITSLRE homologous region in the endostatin protein (SEQ ID NO: 5).

FIG. 6 shows the staining of live endothelial cells.

FIG. 7 shows the staining of fixed endothelial cells

### DETAILED DESCRIPTION

The following description includes the best presently contemplated mode of carrying out the invention. This description is made for the purpose of illustrating the general principles of the inventions and should not be taken in a limiting sense. All publications, references, applications and patents listed or cited herein are incorporated by reference in their entirety.

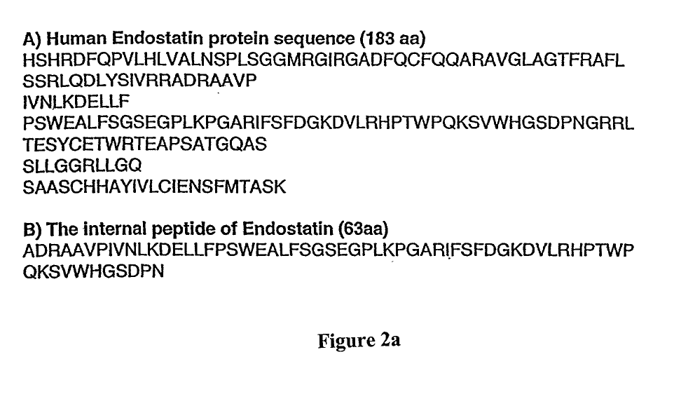

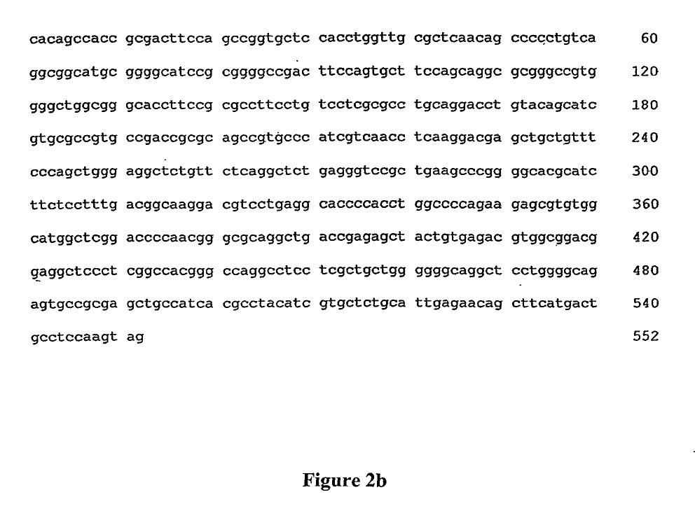

As used herein, the term “endostatin” refers to an antiangiogenic fragment of a most C-terminal non-collagenous region of a collagen protein. It is to be understood that the terms “endostatin” and “endostatin protein” are equivalent and interchangeable. In a preferred embodiment, the C-terminal noncollagenous region is an NC1 region. The collagen protein may be any collagen protein (any member of the collagen family of proteins), but is preferably a non-fibrillar collagen protein, and more preferably a collagen XVIII, a collagen XV or a collagen IV. In one embodiment, an endostatin is a fragment of an approximately 35 kDa C-terminal non-collagenous region of collagen XVIII. In a further embodiment, an endostatin is a fragment of an approximately 35 kDa C-terminal non-collagenous region of collagen XVIII having a molecular weight of between approximately 18 kDa and 20 kDa. In another embodiment, an endostatin is a fragment of an approximately 35 kDa C-terminal non-collagenous region of collagen XVIII and comprises an amino acid sequence identical or substantially homologous to amino acids 1105 to 1124 of mouse collagen alpha 1 type XVIII. In another embodiment, an endostatin is a fragment of an approximately 35 kDa C-terminal non-collagenous region of collagen XVIII and comprises an amino acid sequence identical or substantially homologous to amino acids 1132 to 1151 of human collagen alpha 1 type XVIII. One preferred amino acid sequence for human Endostatin protein is shown in FIG. 2 and SEQ ID NO: 1.

As used herein, the term “angiogenesis-related protein” refers to endostatin protein, and antiangiogenic fragments and homologs thereof. The term “angiogenesis-related protein” includes proteins that are animal or human in origin and also includes proteins that are made synthetically by chemical reaction, or by recombinant technology in conjunction with expression systems. Angiogenesis-related proteins can be isolated from body fluids including, but not limited to, serum, urine and ascites, or synthesized by chemical or biological methods (including cell culture, recombinant gene expression, peptide synthesis). The proteins may also be obtained by in vitro enzymatic catalysis of collagen to yield active endostatin protein. Recombinant techniques include gene amplification from DNA sources using the polymerase chain reaction (PCR), and gene amplification from RNA sources using reverse transcriptase/PCR. General references for methods that can be used to perform the various PCR and cloning procedures described herein can be found in Molecular Cloning: A Laboratory Manual (Sambrook et al., eds. Cold Spring Harbor Lab Publ. 1989, latest edition).

The terms “a”, “an” and “the” as used herein are defined to mean “one or more” and include the plural unless the context is inappropriate. As employed herein, the phrase “biological activity” refers to the functionality, reactivity, and specificity of compounds that are derived from biological systems or those compounds that are reactive to them, or other compounds that mimic the functionality, reactivity, and specificity of these compounds. Examples of suitable biologically active compounds include enzymes, antibodies, antigens and proteins.

The term “bodily fluid,” as used herein, includes, but is not limited to, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, and vitreal fluid, and nasal secretions.

The phrases “isolated” or “biologically pure” refer to material which is substantially or essentially free from components which normally accompany it as found in its native state.

As used herein, the term “protein” refers to full length proteins, active fragments thereof, peptides, mimotopes, structural analogs, and corresponding amino acid sequences.

An “amino acid residue” is a moiety found within a protein or peptide and is represented by —NH—CHR—CO—, wherein R is the side chain of a naturally occurring amino acid. When referring to a moiety found within a peptide, the terms “amino acid residue” and “amino acid” are used interchangeably. An “amino acid residue analog” includes D or L configurations having the following formula: —NH—CHR—CO—, wherein R is an aliphatic group, a substituted aliphatic aromatic group, a benzyl group, a substituted benzyl group, an aromatic group or a substituted aromatic group and wherein R does not correspond to the side chain of a naturally occurring amino acid.

Suitable substitutions for amino acid residues in the sequence of the binding peptides and binding proteins described herein include conservative substitutions that result in peptide and protein derivatives that bind angiogenesis-related proteins. Suitable substitutions for amino acid residues in the sequence of the Endostatin proteins described herein include conservative substitutions that result in antiangiogenic Endostatin protein derivatives. A conservative substitution is a substitution in which the substituting amino acid (naturally occurring or modified) is structurally related to the amino acid being substituted. “Structurally related” amino acids are approximately the same size and have the same or similar functional groups in the side chains.

Provided below are groups of naturally occurring and modified amino acids in which each amino acid in a group has similar electronic and steric properties. Thus, a conservative substitution can be made by substituting an amino acid with another amino acid from the same group. It is to be understood that these groups are non-limiting and that additional modified amino acids could be included in each group.

Group I includes leucine, isoleucine, valine, methionine and modified amino acids having the following side chains: ethyl, n-propyl n-butyl. Preferably, Group I includes leucine, isoleucine, valine and methionine.

Group II includes glycine, alanine, valine and a modified amino acid having an ethyl side chain. Preferably, Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, —NH₂, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, —CO—NH-alkylated glutamine or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, methyl aspartate, ethyl aspartate, benzyl aspartate and methyl glutamate, ethyl glutamate and benzyl glutamate, glutamine and asparagine.

Group V includes histidine, lysine, omithine, arginine, N-nitroarginine, β-cycloarginine, γ-hydroxyarginine, N-amidinocitruline and 2-amino-4-guanidinobutanoic acid, homologs of lysine, homologs of arginine and homologs of ornithine. Preferably, Group V includes histidine, lysine, arginine and omithine. A homolog of an amino acid includes from 1 to about 3 additional or subtracted methylene units in the side chain.

Group VI includes serine, threonine, cysteine and modified amino acids having C1-C5 straight or branched alkyl side chains substituted with —OH or —SH, for example, —CH₂CH₂OH, —CH₂CH₂CH₂OH or —CH₂CH₂OHCH₃. Preferably, Group VI includes serine, cysteine or threonine.

In another aspect of the present invention, suitable substitutions for amino acid residues in the amino acid sequences described herein include “severe substitutions” that result in binding peptides and binding proteins that bind to angiogenesis-related proteins. Suitable substitutions for amino acid residues in the amino acid sequences described herein also include “severe substitutions” that result in Angiostatin or Endostatin protein derivatives that are antiangiogenic. Severe substitutions that result in binding peptide and binding protein derivatives and antiangiogenic Angiostatin or Endostatin protein derivatives are much more likely to be possible in positions that are not highly conserved than at positions that are highly conserved. A “severe substitution” is a substitution in which the substituting amino acid (naturally occurring or modified) has significantly different size and/or electronic properties compared with the amino acid being substituted. For example, the side chain of the substituting amino acid can be significantly larger (or smaller) than the side chain of the amino acid being substituted and/or can have functional groups with significantly different electronic properties than the amino acid being substituted.

Examples of severe substitutions of this type include the substitution of phenylalanine or cyclohexylmethyl glycine for alanine, isoleucine for glycine, a D amino acid for the corresponding L amino acid or —NH—CH[(—CH₂)₅—COOH]—CO— for aspartic acid. Alternatively, a functional group may be added to the side chain, deleted from the side chain or exchanged with another functional group. Examples of severe substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties that the functional group of the amino acid being substituted. Examples of such modifications include tryptophan for glycine, lysine for aspartic acid and —(CH₂)₄COOH for the side chain of serine. These examples are not meant to be limiting.

The present invention encompasses homologs, orthologs and paralogs of proteins related to endostatin and PITSLRE protein kinases. Homologs are defined as proteins with substantial homology. “Substantial homology” exists between two amino acid sequences when a sufficient number of amino acid residues at corresponding positions of each amino acid sequence are either identical or structurally related such that a protein or peptide having the first amino acid sequence and a protein or peptide having the second amino acid sequence exhibit similar biological activities. Generally, there is substantial sequence homology among the amino acid sequences when at least 70%, more preferably at least 80%, and most preferably at least 90%, of the amino acids in the first amino acid sequence are identical to or structurally related to the second amino acid sequence. Homology is often measured using sequence analysis software, e.g., BLASTIN or BLASTP. The default parameters for comparing the two sequences (e.g., “Blast”-ing two sequences against each other) by BLASTIN (for nucleotide sequences) are reward for match=1, penalty for mismatch=−2, open gap=5, and extension gap=2. When using BLASTP for protein sequences, the default parameters are reward for match=0, penalty for mismatch=0, open gap=11, and extension gap=1. Additionally, paralogs are defined as proteins having non-identical amino acid sequences and similar functional characteristics, wherein the proteins are from the same species. Orthologs are defined as proteins having non-identical amino acid sequences and similar functional characteristics, wherein the proteins are from different species, but wherein the species have a common ancestral origin. Orthologs have at least 30% homology, more preferably at least 40% homology, and most preferably at least 50% homology among the amino acid sequences.

As used herein, the term “mimetic” refers to a proteinaceous or chemical compound that functions in a manner similar to a endostatin protein or PITSLRE protein kinase. A mimetic of endostatin inhibits angiogenesis and a mimetic of PITSLRE protein kinase is involved in cell cycle regulation.

Unless defined otherwise, all technical and scientific terms used herein have the same meaning as commonly understood by one having ordinary skill in the art to which this invention belongs. Although other materials and methods similar or equivalent to those described herein can be used in the practice or testing of the present invention, the preferred methods and materials are now described.

The present invention is related to compositions and methods for the modulation of angiogenesis. In particular, the present invention comprises proteins and mimotopes that are related to, or derived from endostatin, PITSLRE protein kinases and combinations thereof. The proteins of the present invention are capable of modulating angiogenesis through various mechanisms such as the regulation of transcription during the cell cycle or the triggering of the apoptotic signaling pathway. Also included in the present invention are mimotopes.

PITSLRE kinase proteins are believed to be involved in cell cycle regulation. “PITSLRE” represents a specific sequence of amino acids: Proline-Isoleucine-Threonine-Serine-Leucine-Arginine-Glutamine (SEQ ID NO: 6) and although at least 20 isoforms of these protein kinases are known, each of them contains the PITSLRE sequence.

Although the exact mechanism of action is unknown it is postulated that PITSLRE protein kinases influence cytokinesis through transcription and/or the apoptotic pathway. It is thought that PITSLRE protein isoforms are processed by caspase upon death receptor activation and transported into the nucleus to cause abnormal cytokinesis such as mitotic delay (to increase cell doubling time and/or decrease DNA replication). PITSLRE protein kinases appear to be linked to normal regulation of the cell cycle however their role in the regulation of angiogenesis was not contemplated until the present invention.

Until the discovery by the present inventors, PITSLRE protein kinases had not been associated with angiogenesis. Though not wishing to be bound by the following theory, the present inventors propose that that certain anti-angiogenic factors such as endostatin, exert their antiproliferative effects via mechanisms similar to PITSLRE protein kinases. Specifically, the inventors maintain that given certain homology between endostatin and PITSLRE protein kinases, it is likely that these proteins function in a similar manner, for example by binding to similar receptors, activating similar biological pathways. More specifically, it is believed that that endostatin acts like PITSLRE protein kinases by interacting with a protein ‘X’ involved in either the transcription complex during the cell cycle to cause cell cycle arrest, or in apoptotic signaling pathway.

The present invention provides mimotopes comprising proteins that are related to both endostatin and PITSLRE protein kinases, and combinations thereof. Though not wishing to be bound by the following theory, such proteins are believed to be capable of cell cycle regulation by various mechanisms, including but not limited to, exerting their influence on transcription or by triggering apoptotic signaling pathways. The present invention includes both the amino acid and nucleic acid sequences that code for the presently described proteins and mimotopes. Also included are analogs which have the same or similar structural conformations as the mimotopes.

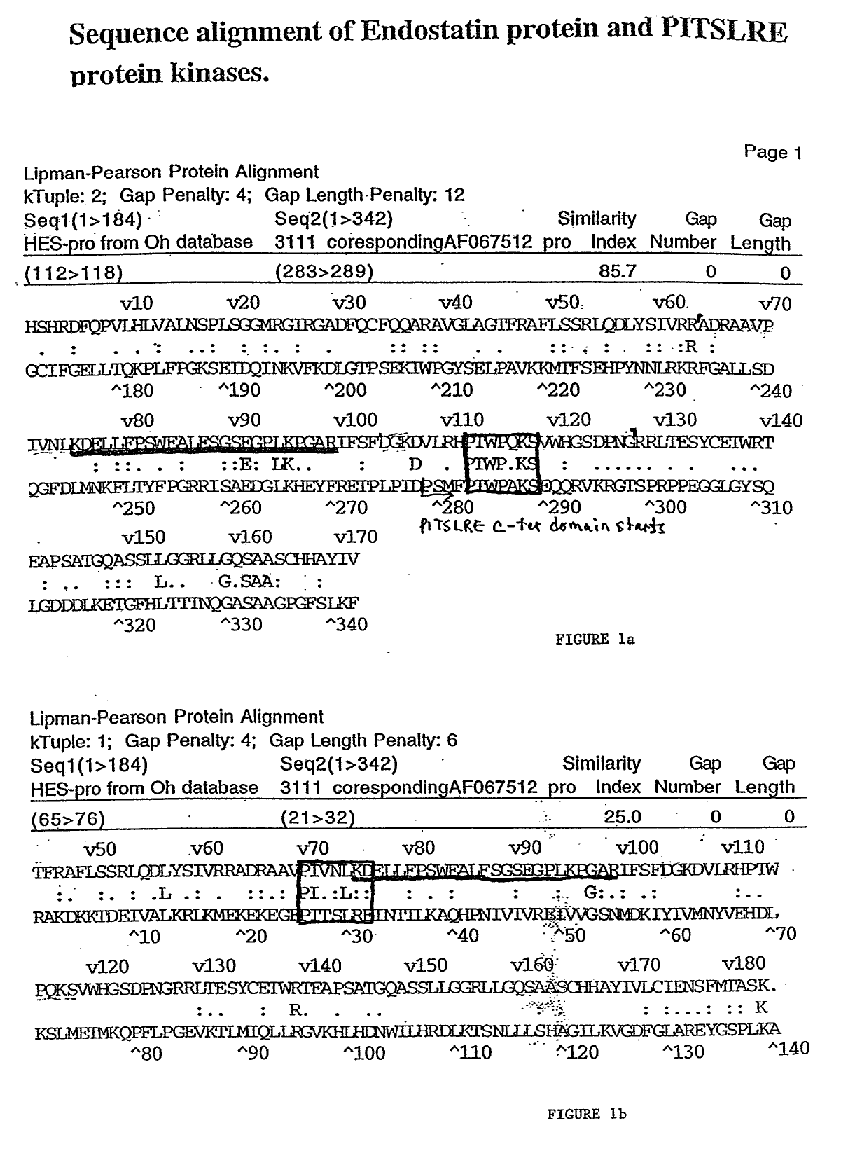

Sequence alignment between a PITSLRE protein kinase cDNA clone and endostatin shows that there are at least two homologous stretches. One stretch in endostatin is a 7 amino acid stretch (PTWPQKS (SEQ ID NO: 7)), 12 amino acids down stream of the peptide used for generating the monoclonal antibody (mAb) 96-2. This first homologous stretch aligns with a stretch of sequence (PTWPAKS (SEQ ID NO: 8)) within PITSLRE protein kinase C-terminal domain (see alignment FIG. 1a). The second homologous stretch in endostatin is a 7 amino acid stretch (PIVNLKD (SEQ ID NO: 9)) very upstream to the peptide used for generating the mAb 96-2. It aligns with a stretch of sequence (PITSLRE (SEQ ID NO: 6)) within the PITSLRE protein kinase catalytic domain (see alignment FIG. 1b).

The present invention further encompasses the use of proteins related to, or derived from, endostatin and PITSLRE protein kinases, such as cell cycle regulating proteins, for the detection of endostatin, in bodily fluids and tissues for the purpose of diagnosis or prognosis of angiogenesis-related diseases. As used herein, the term “angiogenesis-related disease” refers to diseases and conditions including, but not limited to, hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, and cat scratch fever. The present invention also includes methods of treating or preventing angiogenesis-related diseases including, but not limited to, arthritis and tumors by using the compositions of the present invention for cell cycle regulation.

The present invention encompasses compositions and methods comprising nucleotide sequences of preferred peptides and proteins that are related, to or derived from, PITSLRE protein kinases and corresponding homologs. The present invention further encompasses compositions and methods comprising vectors containing nucleotide sequences encoding peptides and proteins derived from PITSLRE, wherein the vector is capable of expressing such binding peptides when present in a cell, and compositions comprising a cell containing such a vector. Because of degeneracy in the genetic code, alternative nucleotide sequences can code for a peptide with the same sequence. The present invention further includes a method comprising, implanting into a human or non-human animal, a cell containing such a vector.

The present invention also encompasses gene therapy whereby genes encoding proteins and peptides that are derived from PITSLRE protein kinases, are regulated in a patient. Various methods of transferring or delivering DNA to cells for expression of the gene product protein, otherwise referred to as gene therapy, are disclosed in Gene Transfer into Mammalian Somatic Cells in vivo, N. Yang, Crit. Rev. Biotechn. 12(4): 335-356 (1992), which is hereby incorporated by reference. Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies.

Methods for treating medical problems with gene therapy include therapeutic strategies such as identifying the defective gene and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene that is derived from PITSLRE protein kinases may be placed in a patient to modify the occurrence of angiogenesis or abnormal cell growth.

Many protocols for transfer of peptide DNA or peptide regulatory sequences are envisioned in this invention. Examples of such technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate the desired peptide in cells not normally expressing the corresponding gene.

Gene transfer methods for gene therapy fall into three broad categories-physical (e.g., electroporation, direct gene transfer and particle bombardment), chemical (lipid-based carriers, or other non-viral vectors) and biological (virus-derived vector and receptor uptake). Gene therapy methodologies can also be described by delivery site. Fundamental ways to deliver genes are familiar to those skilled in the art and include ex vivo gene transfer, in vivo gene transfer, and in vitro gene transfer.

Chemical methods of gene therapy may involve a lipid based compound (such as lipofectins or cytofectins), not necessarily a liposome, to ferry the DNA across the cell membrane. Another chemical method may use receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Biological methods used in gene therapy techniques may involve receptor-based endocytosis, or receptor-based phagocytosis, which involve binding a specific ligand to a cell surface receptor and enveloping and transporting the ligand across the cell membrane. Specifically, a ligand/gene complex is created and injected into the blood stream. Target cells having a receptor for the ligand will specifically bind the ligand and transport the ligand-DNA complex into the cell. Additional biological methods employ viral vectors to insert genes into cells. For example, altered retrovirus vectors have been used in ex vivo methods to introduce genes into peripheral and tumor-infiltrating lymphocytes, hepatocytes, epidermal cells, myocytes, and other somatic cells. These altered cells are then introduced into the individual.

Viral vectors that have been used for gene therapy protocols include, but are not limited to, retroviruses such as murine leukemia retroviruses, RNA viruses such as poliovirus or Sindbis virus, adenovirus, adeno-associated virus, herpes viruses, SV40, vaccinia and other DNA viruses. Replication-defective murine retroviral vectors are the most widely utilized gene transfer vectors. Fundamental advantages of retroviral vectors for gene transfer include efficient infection and gene expression in most cell types, precise single copy vector integration into target cell chromosomal DNA, and ease of manipulation of the retroviral genome. The adenovirus is capable of transducing novel genetic sequences into target cells in vivo. Adenoviral-based vectors express gene product proteins at high levels and have high efficiencies of infectivity, even with low titers of virus. Additionally, the virus is fully infective as a cell free virion so injection of expression cell lines is not necessary. Another potential advantage to adenoviral vectors is the ability to achieve long term expression of heterologous genes in vivo.

Mechanical methods of DNA delivery include direct injection of DNA, such as microinjection of DNA into germ or somatic cells, pneumatically delivered DNA-coated particles, such as the gold particles used in a “gene gun,” inorganic chemical approaches such as calcium phosphate transfection and electroporation. It has been found that injecting plasmid DNA into muscle cells yields high percentage of the cells that are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be re-injected periodically to maintain the gene product level without mutations occurring in the genomes of the recipient cells. Non-integration of exogenous DNAs may allow for the presence of several different exogenous DNA constructs within one cell with all of the constructs expressing various gene products.

Both particle-mediated gene transfer methods and electroporation can be used in in vitro systems, or with ex vivo or in vivo techniques to introduce DNA into cells, tissues or organs. With regard to particle-mediated gene transfer, a particle bombardment device, or “gene gun,” is used that generates a motive force to accelerate DNA-coated high density particles (such as gold or tungsten). These particles penetrate the target organs, tissues or cells. Electroporation mediated gene transfer comprises the use of a brief electric impulse with a given field strength that is used to increase the permeability of a membrane in such a way that DNA molecules can penetrate into the cells.

The gene therapy protocol for transfecting DNA encoding binding peptide and binding proteins into a individual may either be through integration of the binding peptide and binding protein DNA into the genome of the cells, into minichromosomes or as a separate replicating or non-replicating DNA construct in the cytoplasm or nucleoplasm of the cell. Binding peptide and binding protein expression may continue for a long-period of time or the DNA may be re-injected periodically to maintain a desired level of the binding peptide and binding protein in serum or in a cell, tissue or organ.

Compositions of the present invention comprising proteins related to or derived from endostatin and PITSLRE protein kinases, homologs and active fragments thereof, can be prepared in a physiologically acceptable formulation, such as in a pharmaceutically acceptable carrier, using known techniques. For example, the proteins of the present invention may be combined with a pharmaceutically acceptable excipient to form a therapeutic composition.

The therapeutic composition may be in the form of a solid, liquid or aerosol. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic angiogenesis-modulating composition, for example, subcutaneously. Examples of liquid compositions include formulations adapted for injection subcutaneously, intravenously, intra-arterially, and formulations for topical and intraocular administration. Examples of aerosol formulations include inhaler formulations for administration to the lungs.

The composition may be administered by standard routes of administration. In general, the composition may be administered by topical, oral, rectal, nasal or parenteral (for example, intravenous, subcutaneous, or intermuscular) routes. In addition, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either of polylactide, polyglycolide, or polylactide co-glycolide (co-polymers of lactic acid and glycolic acid).

The dosage of the composition will depend on the condition being treated, the particular composition used, and other clinical factors such as weight and condition of the patient, and the route of administration.

The composition may be administered in combination with other compositions and procedures for the treatment of diseases. For example, angiogenesis may be treated conventionally with surgery, radiation or chemotherapy in combination with the administration of the compositions of the present invention, which may also be subsequently administered to the patient to stabilize and inhibit the growth of any residual angiogenesis.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

A monoclonal antibody (mAb 96-2) was generated (provided by T. Boehm, Children's Hospital, Boston, Mass.) using the sequence of the peptide located in between endostatin amino acid 75 through 99 (tf-CKDELLFPSWEALFSGSEGPLKPGAR-NH₂ (SEQ ID NO: 10)).

### EXAMPLE 2

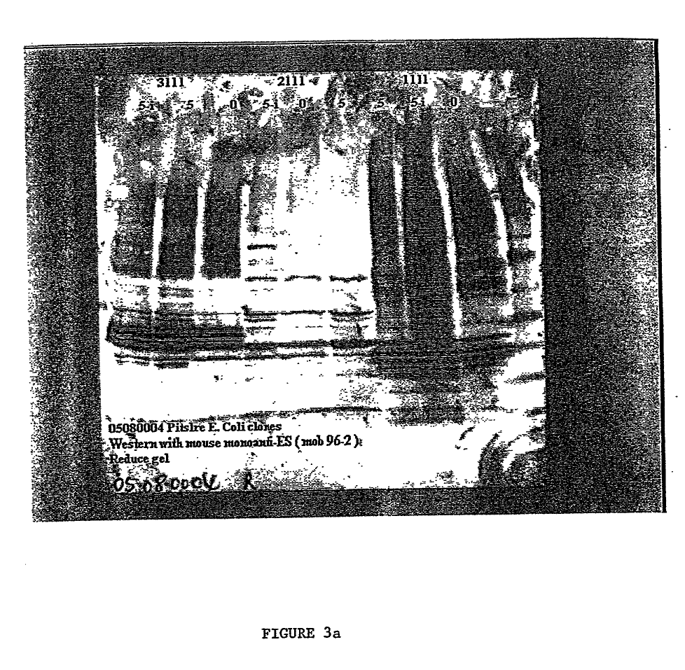

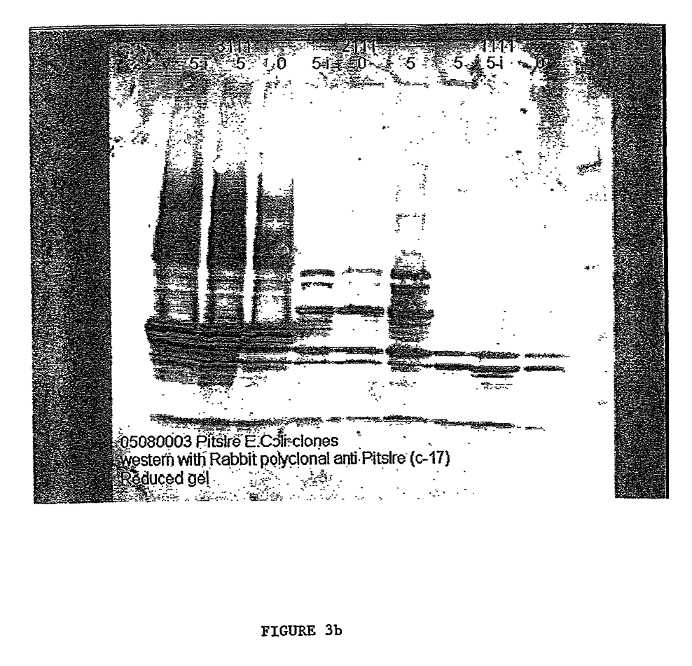

The monoclonal antibody from Example 1 was used to screen the HUVECs Uni-ZAP XR lamda phage expression library, from 200,000 plaques, 7 positives were obtained. Sequence analysis showed that 5 out of 7 positive clones were collagen XIII C-terminal fragments where the endostatin is derived from the other 2 clones (clone 2111 and clone 3111) were PITSLRE protein kinases. These two cDNA clones were specifically recognized by the human endostatin mAb because they have different length of cDNA insert indicating that they were isolated by two separate and independent events. In addition to that, when the E. coli lysate of the two cDNA clones were analyzed on Western Blot probed with the human endostatin mAb 96-2, the bands representing the PITSLRE proteins could also be recognized by the human endostatin monoclonal antibody (FIG. 3a). The proteins recognized by the mAb 96-2 are PITSLRE protein kinases as confirmed by Western blot probed with antiPITSLRE protein kinase antibodies (Santa Cruz Biotechnology, Inc.) (FIG. 3b). These results suggested that human endostatin and PITSLRE protein kinases share an identical mimotope at the human endostatin mAb 96-2 peptide region which can be recognized by the human endostatin mAb 96-2. The PITSLRE protein kinase homologous regions and epitope used for generating mAb 96-2 are within the 63 amino acid endostatin internal sequence of FIG. 2.

### EXAMPLE 3

Sequence alignment between the PITSLRE protein kinase cDNA clone and endostatin shows that there are two homologous stretches. One stretch in endostatin is a 7 amino acid stretch (PTWPQKS (SEQ ID NO: 7)), 12 amino acids down stream of the peptide used for generating the mAb. It aligns with a stretch of sequence (PTWPAKS SEQ ID NO: 8)) within PITSLRE protein kinase C-terminal domain (see alignment FIG. 1a). The other stretch in endostatin is a 7 amino acid stretch (PIVNLKD (SEQ ID NO: 9)) very upstream to the peptide used for generating the mAb. It aligns with a stretch of sequence (PITSLRE) within the PITSLRE protein kinase catalytic domain (see alignment FIG. 1b).

### EXAMPLE 4

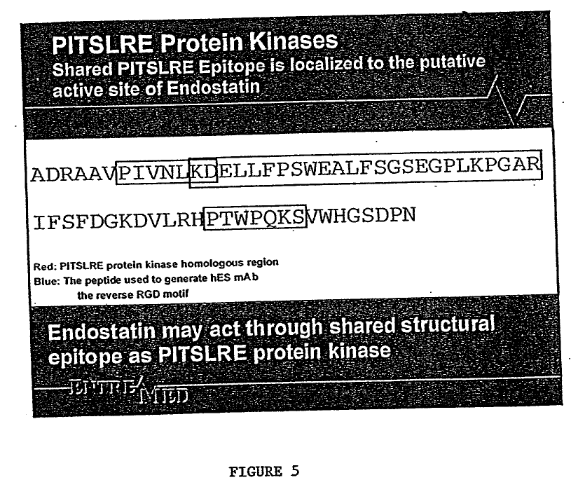

The monoclonal antibody raised against human endostatin peptide 75-99 (SEQ ID NO: 10) recognizes the PITSLRE protein kinase. But the PITSLRE protein kinase does not have any sequence homologous to the peptide used for generating mAb 96-2, it is homologous to the peptide flanking sequences. This indicates that the PITSLRE protein kinases have a mimotope which is identical to the one in endostatin which can be recognized by the endostatin mAb 96-2. In endostatin, this mimotope is located within the 63 amino acid sequence (FIG. 2 and FIG. 5). In the PITSLRE protein kinases, this mimotope could be conformed with any or both of the PTWPAKS (SEQ ID NO: 8) and PITSLRE (SEQ ID NO: 6) sequences and other sequences within the PITSLRE protein kinases which can be recognized by the human endostatin mAb 96-2.

### EXAMPLE 5

### Localization of Endostatin into Cells and Nuclei

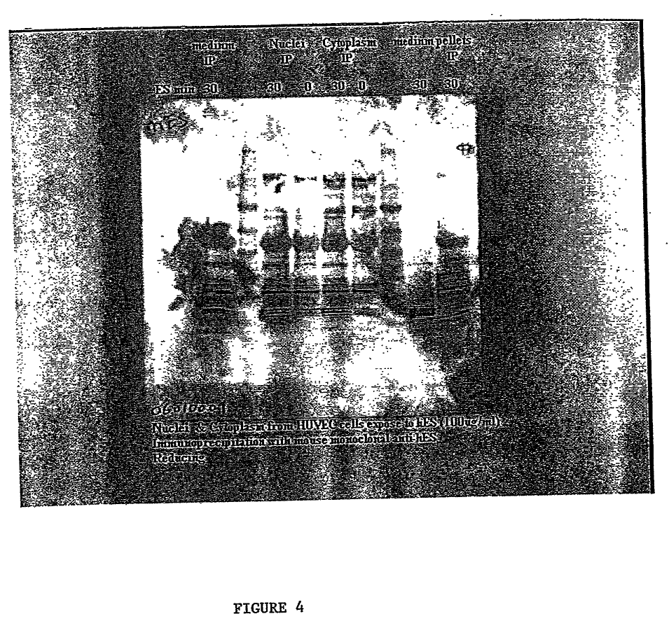

3 T-75 of HUVECs grown at regular growth condition (about 95% confluent) were treated with 100 ug/ml recombinant endostatin for 30 minutes and rinsed with warm 1XPBS and then trypsinized. The control cells from 3 T-75 were the HUVECs treated with the same concentration of endostatin for 1 second. The trypsinized cells were then washed with cold PBS twice. About 40 μl of cell pellet was obtained from all three T-75 flasks. The cytosol extract and nuclei extract was obtained using a nuclei extraction kit (Pierce Company, Rockford, Ill.). The rabbit polyclonal antibodies against human endostatin (Cytimmune Sciences, College Station, Md.) were used to immunoprecipite endostatin protein present in the extract. The immunoprecipited elution was analyzed on Western blot probed with either the same polyclonal antibodies or the mAb 96-2. The results of this experiment demonstrate that endostatin protein is able to localize into HUVEC cell and nuclei and is not further processed to smaller fragments. (FIG. 4).

### EXAMPLE 6

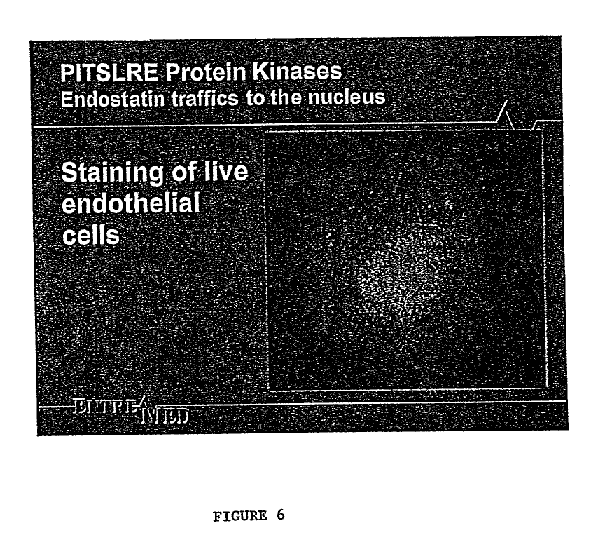

Live HUVECs cells were treated with 8 ug/mI Alexa-labeled endostatin for 5 minutes, 15 minutes, 30 minutes and 60 minutes, land washed with M-200 medium and fixed in 3.7% formaldehyde for 5 minutes and cold methanol for 5 minutes. Alexa-labeled endostatin can be visualized under the fluorescent microscope. The results showed that endostatin stains the cytosol and nuclei even as early as at the 5 minute time point. Staining was even more pronounced in the later time points suggesting that endostatin can access cells even the nuclei (FIG. 6).

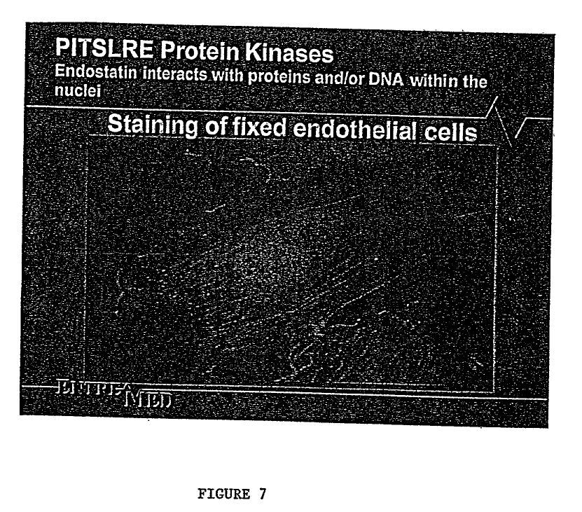

HUVECs grown at 5 ng/ml VEGF were fixed in 3.7% formaldehyde for 5 mins. and cold methanol for 5 mins. and then stained with 40 μg/μl Alexa-labeled endostatin for 30 mins. The results showed that endostatin stained not only the cytoskeleton network, but also stained the nuclei suggesting that there are proteins in both the cytosol compartment and nucleus compartment that endostatin interact with (FIG. 7).

### EXAMPLE 7

The monoclonal antibody raised against human endostatin peptide 75-99 (SEQ ID NO: 10) as described in Example 1 recognizes the PITSLRE protein kinase, but while the PITSLRE protein kinase does not have any sequence homologous to the peptide used for generating mAb 96-2, it is homologous to the peptide's flanking sequences in endostatin. This indicates that the PITSLRE protein kinases have a conformational region which is identical to a conformational structure designated P. Thus, mAb 96-2 recognizes conformational region P. This mAb 96-2 also recognizes endostatin and was raised against endostatin peptide region (75-99 (SEQ ID NO: 10)) indicating that endostatin contains the confoinational structure P.

Although not wanting to be bound to the following hypothesis, it is believed that this conformational region P, including the two PITLSRE homologous regions and the peptide 75-99 (SEQ ID NO: 10) of endostatin, is the functional epitope of endostatin. The 63-mer and 31-mer endostatin peptides of FIG. 5 have been shown to have potent tumor inhibition activities in vivo in their Xenografts model. Both sequences contain the 75-99 peptide and PTWPQKS domain (SEQ ID NO: 7); the 63-mer peptide contain the PIVNLKD domain (SEQ ID NO: 9) also.

It should be understood that the foregoing relates only to preferred embodiments of the present invention, and that numerous modifications or alterations may be made therein without departing from the spirit and the scope of the invention as set forth in the appended claims. The references cited throughout are hereby incorporated by reference in their entireties.

1. A method of regulating angiogenesis comprising administering to a human or animal a composition comprising a protein that is homologous to PITSLRE protein kinase and an angiogenic factor.

2. The method of claim 1, wherein the angiogenic factor is endostatin.

3. The method of claim 1, wherein angiogenesis is related to cancer, arthritis, macular degeneration, and diabetic retinopathy.

4. The method of claim 1, wherein the protein comprises the amino acid sequence set forth in SEQ ID NO: 7.

5. The method of claim 1, wherein the protein is homologous to the amino acid sequence set forth in SEQ ID NO: 8.

6. The method of claim 1, wherein the protein comprises the amino acid sequence set forth in SEQ ID NO: 9.

7. The method of claim 1, wherein the protein is homologous to the amino acid sequence set forth in SEQ ID NO: 6.

8. A method of regulating angiogenesis comprising administering to a human or animal a composition comprising a PITSLRE protein kinase and active fragments thereof.

9. The method of claim 9 wherein angiogenesis is decreased.

10. The method of claim 8, wherein angiogenesis is related to cancer, arthritis, macular degeneration, and diabetic retinopathy.

11. The method of claim 8, wherein the protein comprises the amino acid sequence set forth in SEQ ID NO: 7.

12. The method of claim 8, wherein the protein is homologous to the amino acid sequence set forth in SEQ ID NO: 8.

13. The method of claim 8, wherein the protein comprises the amino acid sequence set forth in SEQ ID NO: 9.

14. The method of claim 8, wherein the protein is homologous to the amino acid sequence set forth in SEQ ID NO: 6.

15. The method of claim 8, wherein the composition further comprises a pharmaceutically acceptable excipient, carrier or sustained-release matrix.

16. A composition for inhibiting angiogenic-related disease comprising a PITSLRE protein kinase and an angiogenic factor.

17. The composition of claim 16, wherein the angiogenic factor comprises endostatin.

18. The composition of claim 16, wherein the PITSLRE protein kinase is selected from the group consisting of SEQ ID NOS: 7, 8. 9 and 10.

19. The composition of claim 16, further comprising a pharmaceutically acceptable excipient, carrier or sustained-release matrix.

20. The composition of claim 16, wherein the angiogenic-related disease is selected from the group consisting of cancer, arthritis, macular degeneration, and diabetic retinopathy.
